(19) United States
(12) Patent Application Publication
(10) Pub. No.: US 2026/0063531 A1
(43) Pub. Date: Mar. 5, 2026

(54) IN-LINE SPECTROSCOPIC CYTOMETRY FOR REAL-TIME MONITORING OF CELLLULAR AND/OR MOLECULAR BIOPROCESSES

(71) Applicant: physical Sciences, Inc., Andover, MA (US)

(72) Inventors: Jesung Park, Westford, MA (US); Youbo Zhao, Westford, MA (US); William J. Kessler, Groton, MA (US)

(21) Appl. No.: 19/311,089

(22) Filed: Aug. 27, 2025

Related U.S. Application Data
(60) Provisional application No. 63/688,981, filed on Aug. 30, 2024.

(51) Int. Cl. G01N 15/1429; C12M 1/34; G01N 15/1434; G01N 33/50

(52) U.S. Cl. CPC G01N 15/1431; C12M 41/46; G01N 15/1434; G01N 33/5005

(57) ABSTRACT

An analytical method and system for monitoring cellular status wherein excitation energy is focused into an excitation cytometry volume within a cell sample that is located within a bioreactor or fermenter to induce optical signals from intracellular compounds. The resulting optical signals are directed to a detection subsystem that has at least two detection channels. One channel detects the elastic (Rayleigh or Mie) scattering signal from the cell that identifies the presence of the cell within the excitation volume. Another channel detects the fluorescence and/or Raman scattering signal of intracellular compounds of the cells. The elastic scattering signals associated with individual cells are used as event-triggers to gate the detection of the intracellular fluorescence and/or Raman signal to eliminate the background noises from the suspension media. The intracellular fluorescence and/or Raman scattering signal are used to characterize cellular status based on the fluorescence intensities at specific fluorescence wavelengths and peak analysis of Raman scattering spectra.

### RELATED APPLICATIONS

This application claims benefit of and priority to U.S. Provisional Application Ser. No. 63/688,981 filed Aug. 30, 2024, under 35 U.S.C. §§ 119, 120, 363, 365, and 37 C.F.R. § 1.55 and § 1.78, which is incorporated herein by this reference.

### GOVERNMENT RIGHTS

This invention was made with U.S. Government support under Contract Nos. 1R43EY029927, 1R43GM150354-01, and 1R43GM154541-01 awarded by the US National Institutes of Health. The Government has certain rights in the subject invention.

### FIELD OF THE INVENTION

Disclosed is an apparatus and method for the real-time measurement of the properties within or at the surface of particles (e.g., microorganisms, cells, or microvesicles) in suspensions (e.g., cell culture media or body fluids) at the single-particle level, and distinguishing particle signals from background noise associated with the surrounding fluid.

### BACKGROUND OF THE INVENTION

Optical measurement of the characteristics of flowing particles in suspensions at the single-particle level pertains to a wide range of applications. These include, but are not limited to, counting and characterizing blood cells in plasma samples, monitoring physiological changes of cells during cultivation, and detecting microbial contamination in cell suspensions. Due to the rapid motion of particles in suspension, single-particle optical measurements are inherently challenging particularly when particle-associated optical signals such as fluorescence or Raman scattering signals are weak and masked by strong background noise from the surrounding fluid.

Bioreactors are specialized systems engineered to cultivate and maintain cells and living organisms under optimally controlled environmental conditions. Among them, agitated bioreactors are extensively employed in the production of a wide range of biologic pharmaceuticals, including but not limited to natural product-based medicines, vaccines, antibiotics, enzymes, proteins, viruses, and cell-based therapies. Recent advances in bioreactor design and bioprocess development have significantly transformed the biotechnology industry enabling the discovery of novel therapeutics and enhancing both the productivity and quality of biologics manufacturing. Consequently, bioprocess development has emerged as a dynamic and strategically important area of investment within the biotechnology and pharmaceutical sectors.

The inherent complexity of cell culture bioprocesses coupled with the sophisticated nature of the resulting biologic products presents substantial challenges in bioreactor development and operational control. Precise characterization and regulation of cell culture conditions are essential to maintain host cell viability and functionality thereby supporting the efficient synthesis of biologic products. Accordingly, real-time process monitoring tools capable of delivering accurate and timely information on cellular physiological states and molecular composition are important for the effective control and optimization of bioreactor performance.

Conventional electronic and optical sensors have been successfully utilized to monitor key process parameters such as pH, pressure, temperature, and concentrations of various metabolites including dissolved oxygen (DO), dissolved carbon dioxide (DCO₂), glucose, glutamine, and lactate. These in-line monitoring systems typically rely on extracellular measurements in conjunction with metabolic flux modeling to infer the physiological status of the cultured cells. However, such indirect approaches often fall short of providing detailed real-time insights at the cellular level limiting their ability to support nuanced control of cultivation conditions and product quality.

Alternatively, offline analyses are commonly employed to assess cell physiology at both the cellular and molecular levels. These methods include mass spectrometry, optical spectroscopy, microscopy, and various forms of cytometry. While these techniques can offer detailed insights, they require the extraction of culture samples at discrete time points and typically involve significant delays between sampling and data acquisition thereby limiting their utility for real-time process control. Furthermore, the necessity for complex, labor-intensive sample preparation procedures introduce variability and increases the risk of altering the native physiological state of the cells raising concerns about the accuracy and relevance of the data with respect to actual bioreactor conditions. In certain bioprocess applications, repeated sampling can also introduce a significant risk of contamination potentially leading to batch failure and substantial economic losses.

More effective in-line process analytical technology (PAT) tools support the growing demands of the biopharmaceutical industry. For instance, Ovizio has introduced the iLine F microscope—a commercial platform for real-time, continuous bioprocess monitoring. While this reflection-based microscopy system offers sub-cellular resolution and enables observation of cell morphology, it lacks the capability to capture critical functional and molecular information necessary for comprehensive cell characterization. In a 2024 publication Gupta, et al. (Microsystems & Nanoengineering (2024) 10:35) combined scatter detection with fluorescence measurements to identify when a cell was present in the detection path to monitor contents within the cell. Their measurement approach required sampling and the use of a chip structure with a central channel to enable the cytometry measurements. Research efforts have also explored the integration of cytometry technologies into bioreactor systems. However, these approaches typically require extensive modifications to existing bioreactor infrastructure creating substantial barriers to industrial adoption.

### SUMMARY OF THE INVENTION

An objective of the present invention is to provide a method and apparatus for real-time continuous monitoring of cellular redox metabolism and/or the identification of cellular biomolecules at both the cellular and molecular levels within agitated bioreactors used in biopharmaceutical manufacturing using the natural flow dynamics of the reactor to enable cytometry-like measurements. This invention aims to enhance process control thereby improving the productivity and quality of biologic products. The disclosed technology addresses this challenge through a dual-channel, event-triggered detection approach. While this technology is demonstrated primarily for characterizing cell physiology in bioreactor cultures in this disclosure, the methodology is broadly applicable across the use cases. Disclosed is an apparatus and method for the real-time measurement of the properties within or at the surface of particles (e.g., microorganisms, cells, or microvesicles) in suspensions (e.g., cell culture media or body fluids) at the single-particle level, and distinguishing particle signals from background noise associated with the surrounding fluid. More specifically, in certain embodiments, it relates to an integrated system designed for monitoring biological properties of cells (e.g., redox metabolism) at the cellular level and/or detecting biomolecular components in cells cultured within agitated bioreactors used in the production of biologic therapeutics including monoclonal antibodies, viral vectors for gene therapy and natural products. Other embodiments may involve real-time, non-destructive characterization of cellular properties in blood samples, assessment of microorganism integrity in culture media, and analysis of microvesicle dynamics (e.g., lyophilized beads and macrovesicles in culture media or body fluids).

In one embodiment, the present invention provides a method and apparatus for real-time measurement of cellular redox metabolism and/or identification of cellular biomolecules using an in-line spectroscopic cytometry setup. The in-line spectroscopic cytometry setup is preferably configured to detect the fluorescence and/or Raman spectroscopic signals emitted by key metabolic cofactors including flavin adenine dinucleotide (FAD), reduced nicotinamide adenine dinucleotide/phosphate [NAD(P)H], and/or by key biomolecules including deoxyribonucleic acid (DNA) and protein when excited by light absorption. The system can be further configured to quantify a cellular redox ratio on the detected autofluorescence and/or Raman spectroscopic signals. Additionally, the in-line spectroscopic cytometry setup can be further configured to enable real-time identification of cellular biomolecules, including but not limited to, DNA and proteins. In one example, this is achieved through the detection and analysis of Raman scattering signals specific to the molecular composition of the cells. The integrated spectroscopic cytometry system provides a non-invasive and continuous method for monitoring cellular physiology and molecular profiles within bioreactor environments during bioprocess operations.

The bioreactor in-line spectroscopic cytometry technology for real-time measurements of cellular-level reduction-oxidation (redox) metabolism has been demonstrated during vial vector processes using HEK 293 cells. This innovative in-line spectroscopic cytometry system utilizes a redox probe configuration that assesses cellular metabolism by measuring single-photon excited (SPE) autofluorescence spectra emitted from NAD(P)H and FAD, two essential metabolic coenzymes involved in primary cell energy pathways. The ratio of the autofluorescence intensities of NAD(P)H and FAD provides an indicator of the intracellular redox state by reflecting the relative concentrations of these coenzymes. Event-triggered signal detection within the bioreactor, leveraging both the natural flow and the subcellular, tightly confined light excitation and light signal detection design enables cytometry-like measurement of fluorescence emissions spatially confined to sub-cellular volumes resulting in enhanced spatial resolution and a significant reduction in background noise interference. Although there have been prior reports of triggered detection, using target scattering to verify a fluorescence signal is originating from a cell, no reports have been made of using the natural flow dynamics of cell suspensions in agitated bioreactors to enable continuous in-line cytometric measurements during bioprocessing. The flow within bioreactors and fermenters is typically created by a mixing propeller and is used to continuously change the environment surrounding the cells to provide dissolved oxygen, carbon dioxide and nutrients to the cells to maintain cell health and support production of desired cell products. Our new approach takes advantage of this cellular flow within a bioreactor or fermenter by using a specially designed probe that creates a subcellular optically-based cytometry volume in the bioreactor or fermenter itself. This approach eliminates the need for sample extraction and enables real-time process control using prompt analytical measurements. The event-based triggering approach solves the background noise problem of signal emanating from cell media rather than the cells themselves.

During bioreactor culture processes involving HEK 293 cells, the system successfully captured metabolic transitions corresponding to the shift from the cell growth phase to the production phase. These observations confirm the utility of the system for detecting dynamic physiological changes in real time. In addition to redox metabolism profiling, the in-line cytometry platform also demonstrated the capability for continuous monitoring of other culture parameters, including cell density and viability. Although current implementations have been primarily focused on viral vector production within small-scale bioreactors, the technology is readily adaptable for a broad range of biopharmaceutical manufacturing processes including those for protein therapeutics, cell therapies, and gene therapies. Moreover, the applicability of this system extends beyond the pharmaceutical and natural products sectors to include other industrial fermentation and bioreactor-based processes involving the cultivation of cells, tissues, or organisms, such as the production of biofuels.

Disclosed is an optical apparatus and method for real-time measurement of cellular redox metabolism and/or identification of cellular biomolecules within a bioreactor environment utilizing an in-line spectroscopic cytometry probe. The system enables spectroscopic cytometry measurements by integrating the natural flow dynamics of suspended cells in agitated bioreactor cultures with a highly spatially confined dual-channel event-triggered fluorescence and/or Raman detection process. The laser excitation and fluorescence and/or Raman detection are confined to a sub-cellular focal volume that is substantially smaller than the dimensions of a typical cell. As individual cells traverse this optical excitation region, they generate discrete elastic (such as Rayleigh and Mie) scattering light and fluorescence and/or Raman scattering signals in the temporal traces of the detection channels. The disclosed technology leverages the bioreactor flow dynamics and the temporal correlation between the elastic scattering signal and the fluorescence/Raman signal to enable gated fluorescence and Raman signal detection triggered by the elastic scattering signal. This approach ensures the detection of fluorescence and Raman signal confined within the cells, excluding the background from the culture media. This behavior effectively establishes an intrinsic flow cytometry system wherein cells are randomly and continuously sampled as they move through the excitation volume. The system described thereby enables real-time, non-invasive monitoring of cellular redox metabolism and/or biomolecule identification with single-cell resolution directly within a bioreactor, without the need for sample extraction or external cytometry instrumentation.

Featured is an optical apparatus and method for bioreactor in-line spectroscopic cytometry wherein tight spatial confinement of the event-triggered fluorescence and/or Raman scattering is achieved through the use of a focused laser beam, such as an ultraviolet (UV) laser, in combination with a high numerical aperture (NA) objective lens. The system is configured to focus the excitation laser to a micron- or submicron-scale volume thereby selectively exciting autofluorescence from intracellular metabolic cofactors including NAD(P)H and FAD and Raman scattering from cellular biomolecules including DNA and proteins. This optical configuration produces a spatially confined excitation volume that is significantly smaller than the size of a typical cell. As a result, when a cell passes through the focal region, the system generates distinguishable discrete signal peaks in the temporal profiles of the elastic scattering and fluorescence or Raman signal traces. When triggered by cell-associated elastic scattering, the detection of fluorescence and/or Raman signal provides a high cell-to-background ratio, enhancing the sensitivity and specificity of the in-line cytometric measurements within the bioreactor environment, without the need for sample extraction.

Further disclosed is an optical apparatus and method for bioreactor in-line spectroscopic cytometry wherein tight UV laser excitation is achieved through a high NA objective lens system designed with miniaturized, cost-effective optical components. The compact form factor of the optical probe is specifically engineered to meet the spatial constraints associated with integration into bioreactors for real-time, in-line measurement applications. The high NA and optical performance necessary for efficient excitation and signal detection are accomplished through a custom optical assembly comprising low-aberration achromatic lenses, a high NA aspheric lens, and a half-ball lens. This optical configuration is optimized for high on-axis performance which is sufficient for the intended application while off-axis performance is intentionally de-emphasized as it is not required in the confined excitation geometry of the system. The resulting design enables efficient, high-resolution excitation and detection within a compact and manufacturable optical probe suitable for in-line deployment in bioprocessing environments.

Also disclosed is an optical apparatus and method for in-line cytometry wherein autofluorescence signals from NAD(P)H and FAD are excited using an optimized single ultraviolet (UV) light source, and Raman scattering signals from cellular biomolecules are excited using a deep ultraviolet (DUV) light source. A preferred system comprises two detection channels: a confocal elastic scattering detection channel and a fluorescence/Raman spectroscopic detection channel configured to enable single-cell level measurements within bioreactor environments. In flowing cell suspensions, when an individual cell traverses the laser focal volume, a distinct signal peak is generated in the temporal profile of the confocal elastic scattering detection channel. The confocal detection geometry defines a tightly focused detection region, which effectively suppresses out-of-focus background signals. These single-cell elastic (Rayleigh and/or Mie) scattering signal peaks are utilized as event triggers to synchronize gated acquisition in the fluorescence/Raman spectroscopic detection channel, thereby enabling gated and cell-specific spectral detection and analysis. Data analysis algorithms, including those based on cross-correlation, have been demonstrated to reliably identify and extract cell-specific events from background noise.

Featured is a process analytical method of real-time monitoring of cell status utilizing optical excitation and spectroscopic detection. Excitation energy is focused into a defined excitation cytometry volume within a cell sample, inducing fluorescence of endogenous fluorophores of the cell and/or associated cellular biomolecules. The emitted fluorescence and/or Raman spectroscopic signals are collected and directed to a detection subsystem, where both fluorescence spectra of cellular fluorophores and/or Raman spectra of biomolecular fluorophores are measured. Analysis of the detected spectroscopic signals permits differentiation between optical signals originating from within or at the surface of cells (fluorescence and/or Raman) and media background fluorescence or Raman signals, thereby confirming the presence of a cell within the excitation volume. The metabolic status of the cell is determined by evaluating the autofluorescence intensity at predetermined wavelengths that correspond to the peak emission of specific fluorophores. Implementation of spectral fitting algorithms enables accurate deconvolution of overlapping emission spectra, facilitating the quantitative separation of autofluorescence contributions from NAD(P)H and FAD. Furthermore, Raman spectral analysis at characteristic wavelengths yields Raman peaks corresponding to specific biomolecular signatures. These peaks provide identification of key cellular biomolecules, allowing for biochemical profiling of individual cells. The combination of fluorescence and/or Raman signal analysis supports robust, label-free, and non-invasive characterization of cellular metabolic and biochemical states.

The cell sample may comprise a cell culture contained within a bioreactor. Preferably, the excitation cytometry volume is in the range of approximately 0.01 μm³ to 100 μm³. In one embodiment, the autofluorescence spectral measurements are derived from cellular fluorophores including NAD(P)H and FAD while Raman scattering spectra are utilized to measure multiple biomolecular fluorophores originating from biomolecular components such as DNA and proteins. The method can be executed inline within a bioreactor.

In one example, a laser beam is directed through a probe inserted into the cell culture. The detection subsystem comprises a spectroscopic detection channel integrated within the probe configured to collect fluorescent signals for the fluorescence spectra of the cell and/or Raman spectra of intracellular biomolecules.

Also featured is an in-line analytical system for monitoring cell status comprising a probe configured for insertion into a cell culture. The probe includes a focusing optic for directing excitation energy to a defined excitation cytometry volume within the cell culture, and a collection channel for receiving fluorescence and/or Raman signals. The system further comprises an excitation source optically coupled to the focusing optic of the probe for delivering the excitation energy, and a detection subsystem optically coupled to the collection channel of the probe. The detection subsystem is configured to detect fluorescence spectra from endogenous or induced cellular fluorophores and/or Raman spectra from cellular biomolecules. A signal processor is operatively coupled to the detection subsystem and configured to analyze the detected spectra. The signal processor identifies peaks within the temporal profile of the elastic scattering signal, and generates electronic (or digital) synchronization signal to trigger the detection of fluorescence spectra of the cells at the excitation cytometry volume, and/or characteristic Raman peaks corresponding to specific intracellular biomolecules. These spectra collected synchronously with the presence of the cells are distinguished from background fluorescence, enabling measurement of the cellular status based on the presence and intensity of both fluorescence and Raman spectral components.

Also featured is an in-line cytometry system for monitoring cell status in agitated bioreactors comprising focusing excitation energy to an excitation cytometry volume that is smaller than a cell to fluorescence cells when present at a focal region of the excitation energy, randomly sampling cells that are moving across the excitation volume, and generating fluorescence and/or Raman scattering time traces that contain cells peaks as the cells flow in the agitated bioreactor. Detection of these signals is processed digitally by synchronizing them with the elastic scattering trigger signals produced by the flowing cells.

The subject invention, however, in other embodiments, need not achieve all these objectives and the claims hereof should not be limited to structures or methods capable of achieving these objectives.

### BRIEF DESCRIPTION OF THE SEVERAL VIEWS OF THE DRAWINGS

Other objects, features and advantages will occur to those skilled in the art from the following description of a preferred embodiment and the accompanying drawings, in which:

FIG. 1 is a block diagram depicting the primary components associated with a new spectroscopic cytometry system and method;

FIG. 2 is a schematic view of an example of a bioreactor in-line spectroscopic cytometry system using a spectrometer for either fluorescence or Raman scattering measurements;

FIGS. 3A-3B illustrate a bioreactor in-line fluorescence spectroscopic cytometry measurement principle, (FIG. 3A) schematic showing the conceptual setup and (FIG. 3B) principle of event-based triggering and detection mechanism;

FIGS. 4A-4B illustrate a bioreactor in-line Raman spectroscopic cytometry measurement principle, (FIG. 4A) schematic showing the conceptual setup and (FIG. 4B) principle of event-based triggering and detection mechanism;

FIGS. 5A-5D show different views of an exemplary in-line spectroscopic cytometry probe, (FIG. 5A) sectional view of the optical beam path, (FIG. 5B) sectional view of mechanics, (FIG. 5C) external view of the probe mechanical design, and (FIG. 5D) photographs of a fabricated probe showing the inserted optics on the top and the metal tube at the bottom;

FIGS. 6A-6C illustrate a Zemax optical design and performance simulation of an example in-line cytometry probe, (FIG. 6A) excitation beam path, (FIG. 6B) cross section of the excitation point spread function and (FIG. 6C) spot size diagram across the excitation focus;

FIGS. 7A-7B show an example of confocal reflectance by elastic scattering (FIG. 7A) and fluorescence spectroscopy of the intracellular NADH and FAD (FIG. 7B); Spikes in the confocal channels represent individual cells which are used to generate trigger pulses to synchronize the spectroscopy channel;

FIGS. 8A-8D show an example of data processing for extracting the cellular redox ratio using a skewed Gaussian fitting algorithm applied to mixtures containing various concentrations of NADH and FAD. The original and fitted spectra show a strong overlap in these figures, indicating a close match between the measured data and the model fit (FIG. 8A) NADH: 1000 μmol and FAD: 50 μmol, (FIG. 8B) NADH: 1000 μmol and FAD: 25 μmol, (FIG. 8C) NADH: 1000 μmol and FAD: 12.6 μmol, and (FIG. 8D) NADH: 1000 μmol and FAD: 6.3 μmol;

FIG. 9 shows in-line spectroscopic cytometry measurements of HEK293 cell metabolism in response to rotenone addition indicated by the arrow, processed based on a skewed Gaussian fit (SGF) and a spectral Band Pass filtering approach;

FIGS. 10A-10D show in-line spectroscopic cytometry measurement of HEK293 cellular redox metabolism during a growth bioreactor run without viral transfection; and its correlation with other measurements; and

FIGS. 11A-11D show in-line spectroscopic cytometry measurement of HEK293 cellular redox metabolism during a transfection bioreactor run with viral transfection, and its correlation with other measurements.

### DETAILED DESCRIPTION OF THE INVENTION

Although one or more preferred embodiments of the invention are described below, the invention is not limited to those specific embodiments. It may be embodied in other forms and implemented in various ways without departing from its scope. Accordingly, the invention should not be construed as being limited to the particular structures or arrangements described in the following specification or shown in the accompanying drawings. If only a single embodiment is disclosed, the claims should not be interpreted as being limited to that embodiment. Furthermore, the claims are not to be interpreted narrowly or restrictively unless clearly and convincingly required by the language or context, including an express exclusion, limitation, or disclaimer.

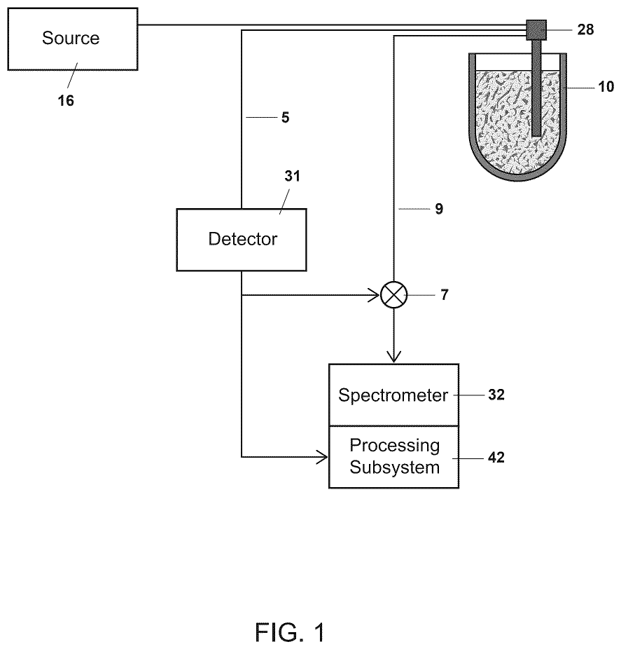

Source 16, FIG. 1 outputs electromagnetic energy to probe 28 in bioreactor 10 stimulating elastic scattering photons and fluorescence and/or Raman photons from a cell in bioreactor 10. A first detection channel 5 is configured to direct elastic scattering photons (signals) only to detector 31 which outputs a trigger signal. Non-cellular material in bioreactor 10 may also generate background scattering signals.

A second detection channel 9 directs fluorescence and/or Raman photons (signals) to a spectroanalysis subsystem, for example, spectrometer 32 and processing subsystem 42 which is responsive to the spectral output of spectrometer and is configured to determine a biochemical profile of the cell from the spectroanalyzed fluorescence and/or Raman photons. For example, a fluorescence redox ratio of the cell can be determined from the spectroanalyzed fluorescence photons. Other biochemical profiles can be determined from the spectroanalyzed fluorescence and/or Raman photons.

There are means for selectively spectroanalyzing the fluorescence and/or Raman photons. In one example, such means includes a shutter 7 in channel 9 triggered by the trigger signal output by detector 31 to only allow the fluorescence and/or Raman photons to reach the spectrometer 32 for spectroanalysis by processing subsystem 42 for so long as the trigger signal is above a predetermined threshold. The shutter can be a motor driven mirror, a digital micromirror device (DMD), a mechanical shutter, or the like.

Alternatively, there is no shutter and the fluorescence and/or Raman photons from a cell always reach the spectrometer but its spectral output is not processed by processing subsystem 42 until processing subsystem 42 receives the trigger signal from detector 31. In this version, selectively analyzing the fluorescence and/or Raman photons includes configuring processing subsystem 42 to be responsive to the trigger signal output by detector 31. Again, the processing subsystem 42 may process the spectrometer output for only so long as the trigger signal output by detector 31 is above a predetermined threshold. As a result, background fluorescence and/or scattering signals from non-cellular material in the bioreactor are reduced for a more accurate analysis.

The bioreactor 10 is commonly employed in pharmaceutical manufacturing and the biotechnology industry. Within the bioreactor, microbial or mammalian cells are cultured under carefully controlled conditions and the delivery of nutrients and critical metabolites is precisely regulated to enhance cellular performance and optimize the synthesis of the desired product. Given the inherent complexity of bioreactor operations, an in-line Process Analytical Technology (PAT) tool depicted in can play a crucial role in characterizing and monitoring cell physiology and culture parameters. This real-time insight supports informed adjustments to feeding strategies and facilitates the advancement of more sophisticated bioprocesses.

The in-line spectroscopic cytometry system is preferably designed to measure elastic (e.g., Rayleigh and Mie) scattering of the cells in order to trigger analysis of autofluorescence and/or Raman scattering signals from key intracellular metabolites such as NAD(P)H and FAD as well as cellular biomolecules including DNA and proteins within agitated bioreactor environments. The synchronized dual-channel measurements enable the assessment of the redox metabolic state at the single-cell level. Specifically, the fluorescence ratio FAD/(FAD+NAD(P)H) is employed to quantify redox states across metabolic pathways. Additionally, distinct fluorophores associated with DNA and protein are used to identify cellular molecules and evaluate their physiological status. The system is further capable of detecting other naturally fluorescing molecules and metabolites by utilizing appropriate combinations of single- or multi-photon laser excitation wavelengths, optical filters, and detection elements. Owing to the inherent spatial and spectral selectivity of photon excitation, the system preferentially measures signals associated with intracellular targets, thereby minimizing interference from compounds present in the surrounding culture media. Leveraging the natural flow dynamics of cells within agitated bioreactor cultures, this approach facilitates real-time, in situ cytometric analysis of cell physiology without the need for sample extraction.

The in-line spectroscopic cytometry system may incorporate a continuous-wave (CW) ultraviolet (UV) and/or deep ultraviolet (DUV) laser source 16 for the detection of autofluorescence and/or Raman scattering but other wavelengths may be used as appropriate.

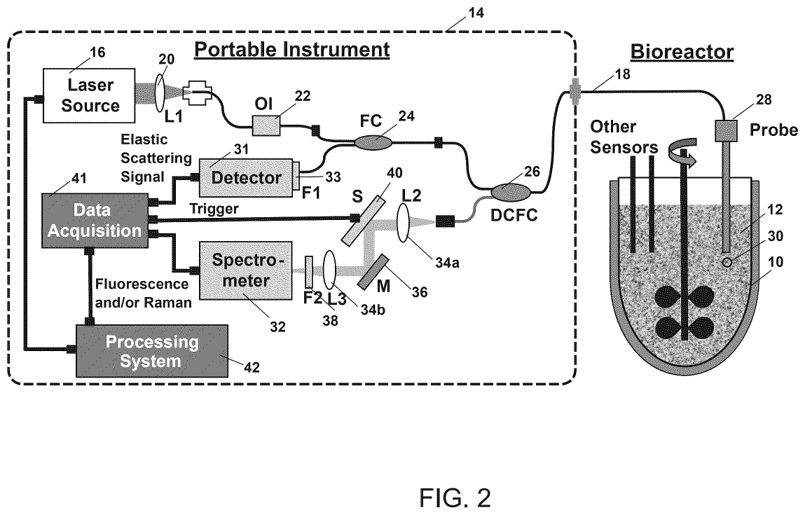

In the example of FIG. 2, the laser light is coupled into a fiber optic 18 using a focusing lens 20 (L1) and passes through an optical isolator 22 (OI) which only allows the light to travel in one direction. The light then passes into a 2×1 fiber coupler 24 (FC). Following the FC the light enters a 2×1 double-clad fiber coupler 26 (DCFC) which transfers the outgoing laser light to probe 28 positioned within the bioreactor 10. Upon exiting the DCFC, the laser beam propagating through the core of the fiber is tightly focused into the cell culture within the bioreactor defining a localized excitation cytometry volume 30. This generates elastic scattering photons and auto fluorescence and/or Raman scattered photons. The elastic scattering photons are directed to detector 31. The autofluorescence and/or Raman scattered photons are directed to spectrometer 32.

The elastic scattering signal of the cells is preferably collected by the core of DCFC 26 and transmitted via FC 24 to the detector 31, FIG. 2. A narrow bandpass filter 33 (F1) is positioned in the detection path to eliminate unwanted wavelength signals (e.g., the fluorescence and/or Raman scattered photons). After passing through the filter, the elastic scattering signal is directed to detector 31 where it is converted into an electrical signal provided to data acquisition system 41 as a trigger signal. The detector is typically a highly sensitive photodetector, such as a photodiode, avalanche photodiode (APD), or photomultiplier tube (PMT). The time-variation elastic signal could also inform the size and morphology of the cell that passes by the excitation volume. The frequency of the cell peaks in the elastic scattering channels correlates with cell density in the solution.

In response to a trigger signal from detector 31, the data acquisition system 41 activates a shutter 40 (S) in synchronization with the elastic scattering signals received from the detector. Now fluorescence and/or Raman photons are directed to spectrometer 32 via the shutter 40 for processing.

The fluorescence and/or Raman scattering signal generated within the laser focal region 30 is preferably smaller than the diameter of individual cells. The emitted intracellular fluorescence and Raman signals are collected through the first cladding of the DCFC 26 and/or a large-core multimode fiber and then directed to spectrometer 32 through a pair of lenses 34a, 34b (L2 and L3), and a reflecting mirror 36 (R). A bandpass filter 38 (F2) is positioned in front of spectrometer 32 to suppress unwanted wavelength noise or enhance signal specificity. The collected signal is then directed to a highly sensitive spectrometer 32 or other spectrally filtered detectors which capture and analyze the fluorescence spectrum across a specified wavelength range. Spectrometer 32 selectively acquires fluorescence and/or Raman signals only when the shutter 40 is engaged and digitizes these signals for subsequent analysis.

The fluorescence and/or Raman signals are converted to electrical signals by the spectrometer 32 and delivered to the data acquisition module 41 and the processing system 42. The module 41 and the processing system 42 can be thought of as components of a processing subsystem. The processing system 42 is configured to execute one or more real-time algorithms to analyze the output of the spectrometer and to extract biological and chemical information from the acquired fluorescence and/or Raman spectral data.

In the fluorescence setup (FIG. 2), the spectrum acquired by the spectrometer 32 is transmitted via data acquisition module 41 to processing system 42, which performs data analysis including spectral fitting to determine NAD(P)H and FAD contributions. The maximum peak intensities at characteristic wavelengths for NAD(P)H and FAD are identified from the fitted spectra. These values are used to calculate the fluorescence redox ratio, defined as FAD/(FAD+NAD(P)H) to assess the redox state and metabolic activity within the bioreactor. For Raman signals, the spectrometer-acquired spectrum is similarly transmitted via data acquisition module 41 and processed by processing system 42 which preferably applies background subtraction and noise-reduction algorithms to enhance peak detection. Distinct Raman peaks are then extracted and correlated with specific biomolecules such as nucleic acids (e.g. DNA), amino acids (e.g. protein) and coenzymes (e.g. NAD) enabling biochemical profiling at the single-cell level.

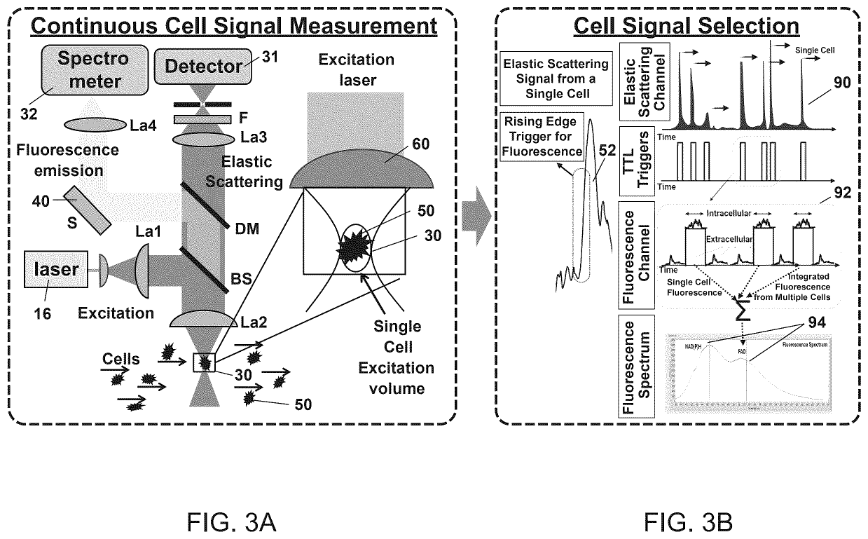

In agitated cell suspension bioreactors, cells flow through the culture media. As illustrated in FIGS. 3A-3B, when a cell 50 crosses the laser focus 30, a distinct signal peak 52 appears in the temporal trace of the elastic scattering signal channel creating an intrinsic cytometry system that randomly samples individual cells. The system utilizes the discrete cell peaks in the elastic scattering channel as the event trigger to synchronize (gate) the analysis of a fluorescence and/or Raman scattering signal enabling efficient discrimination of cell signals from background noise. Gating can be accomplished using physical hardware such as a shutter 40, FIG. 2. Alternatively the gating can be accomplished digitally by simultaneously and continuously acquiring all signal channels and using the temporal synchronization to only process fluorescence and/or Raman signals via the processing subsystem when a cell is in the optical detection path as identified by the presence of the elastic scattering signal produced by the cell. The principle is that a cellular signal peak in the elastic scattering channel is used as a trigger to gate the spectrometer acquisition to selectively detect fluorescence and/or Raman signals from the cells while minimizing the background fluorescence noise from the surroundings. Advanced algorithms, such as cross-correlation, can be employed to identify cellular signal peaks in both the elastic scattering and fluorescence/Raman scattering channels though alternative algorithms may also be applicable.

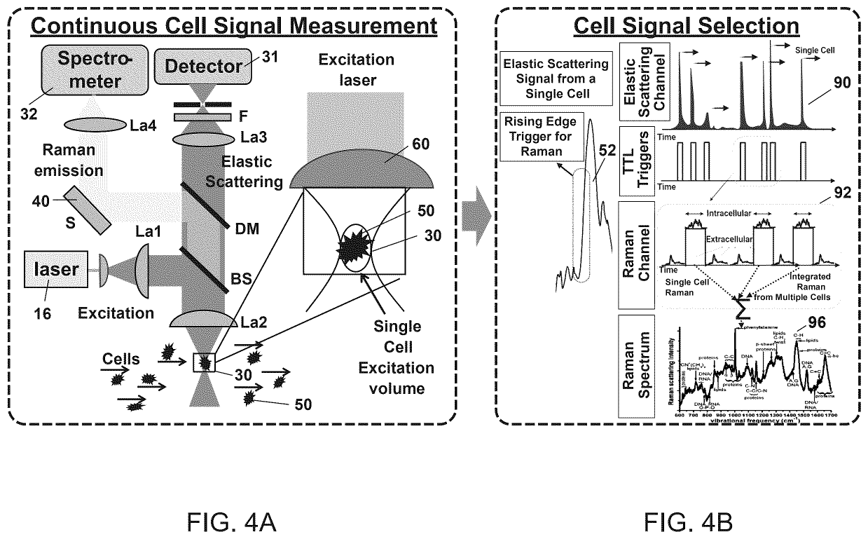

FIGS. 3-4 illustrate a basic schematic configuration of one example of an in-line, real-time spectroscopic cytometry system and the principle of event-based triggering for cell signal selection. This spectroscopic cytometry system includes laser excitation, elastic scattering detection, and fluorescence/Raman spectral detection to detect individual cells as they pass through a defined optical interrogation zone. The laser excitation beam is focused into a small detection volume 30 by a series of relay lenses (La1 and La2) and a beam splitter (BS) enabling excitation of cells within a tightly confined region small enough to isolate optical signals from single cells. As cells pass freothrough this excitation volume, elastic scattering signals are collected and directed to detector 31 via a focusing lens (La3) and an optical filter (F). The detector 31 converts the optical signals to electrical signals. These scattering signals serve as triggers for the data acquisition system marking the presence of a cell within the interrogation zone. Following excitation, the resulting fluorescence and/or Raman emission from the cell is collected and routed through a dichroic mirror (DM) and a focusing lens (La4), and a shutter 40 (S). Acting as a high-speed, programmable optical switch, the shutter directs the emission signals to the spectrometer only when a valid scattering event is detected.

This event-based gating mechanism enables selective and efficient spectral data capture from individual cells. As shown in FIGS. 3A and 4A, excitation energy from laser is focused by optical element 60 into an excitation cytometry volume 30 within a cell sample 50, thereby inducing fluorescence or Raman scattering signals from intracellular fluorophores (e.g., FAD and NAD(P)H enzymes) and cellular biomolecules (e.g., adenine, cytosine, guanine, thymine, and uracil) from DNA and from proteins (e.g., tyrosine, tryptophan, phenylalanine, and methionine). The resulting fluorescence and/or Raman signals are directed to the detection subsystem as illustrated in FIG. 2. An example fluorescence spectrum, shown in FIGS. 3B and 4B, is captured by a spectrometer 32. These fluorescence signals are subsequently acquired by the data acquisition system 41 (FIG. 2) to detect the first peak from NAD(P)H and second peak from FAD within the fluorescence spectrum 94 (FIG. 3B) and/or to identify Raman signatures 96 (FIG. 4B) of cellular biomolecules from the Raman scattering spectrum (FIG. 4B). The elastic signals serve as indicators of the presence of the cell within the excitation cytometry volume effectively distinguishing cellular fluorescence from background signals. Upon detection, cellular parameters such as the redox metabolic state of the cell, and Raman peaks of the cellular biomolecules are analyzed. This analysis may be performed by processing system 42 (FIG. 2). In one example, the unitless redox ratio is determined using the formula FAD/(FAD+NAD(P)H); however, alternative formulations such as FAD/NAD(P)H may also be applied depending on the specific analytical context.

In one embodiment, an in-line analytical system for monitoring cellular redox metabolism includes a probe 28 (FIG. 1) designed for insertion into a cell culture. The probe may incorporate a focusing optic 60 (FIGS. 3 and 4), that directs excitation energy to a defined excitation cytometry volume within the culture. Additionally, the probe includes an elastic scattering channel 90 (FIGS. 3B and 4B) and a fluorescence/Raman collection channel 92 (FIGS. 3B and 4B) for capturing fluorescence and/or Raman signals.

To facilitate real-time, continuous cytometry measurements of cellular properties, a miniaturized optical probe may be inserted into the bioreactor via a sensor port. This probe delivers high optical performance enabling efficient fluorescence and/or Raman excitation and signal detection.

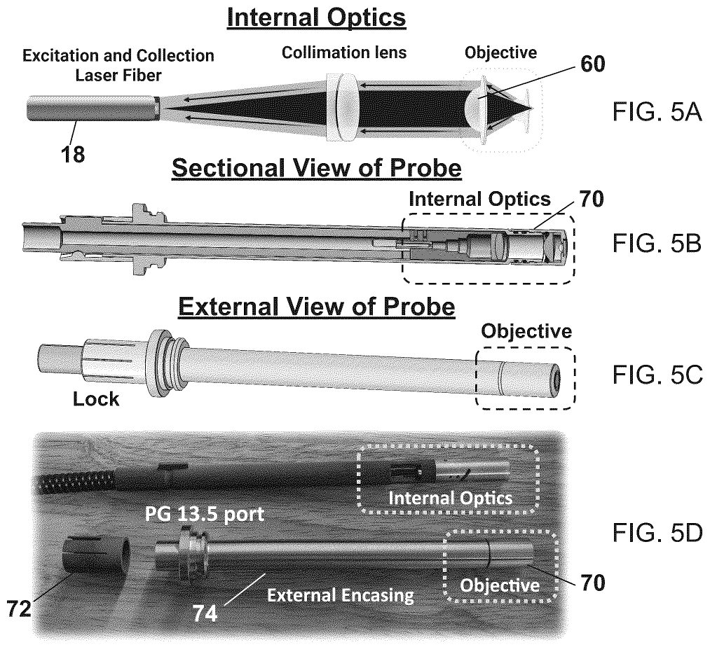

The mechanical design of the developed redox probe is illustrated in FIGS. 5A-5D. The probe includes four primary components: (1) a brass barrel 70 housing the key optical elements; (2) a 3D-printed plastic part 72 that secures the fiber 18 within the brass barrel; (3) a stainless-steel outer tube 74 that encloses the internal components and interfaces with the bioreactor; and (4) a high-numerical-aperture (NA) objective assembly 60. The brass barrel and 3D-printed part can be detachable from the outer tube allowing only the outer tube and objective to remain inside the bioreactor during operation. These remaining components can be autoclavable enabling sterilization without compromising the optics and fibers contained in the brass barrel and plastic part. In one example, the outer tube has an external diameter of 12 mm allowing seamless integration with bioreactors via a standard PG13.5 sensor port.

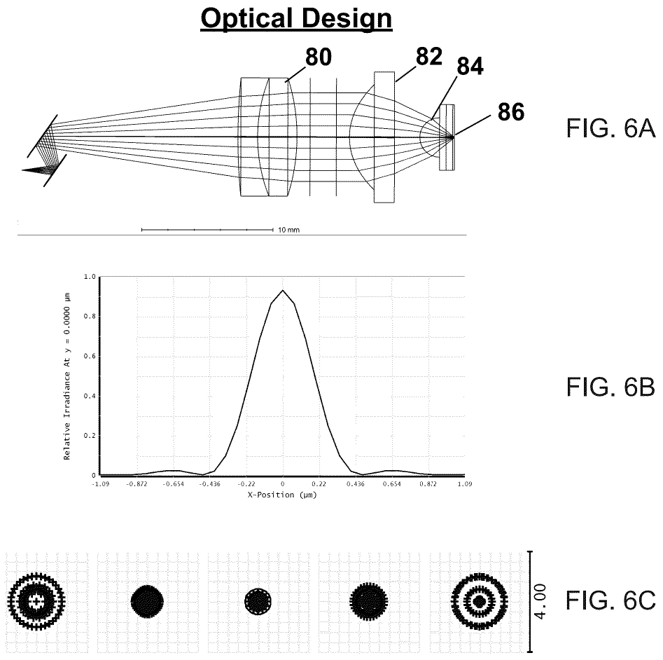

The optical design and performance characteristics of an exemplary probe are illustrated in FIGS. 6A-6C. One component is the high-numerical-aperture (NA) objective 60, which includes an achromatic lens 80 (FIG. 6A), a high-power aspheric lens 82 (FIG. 6A), a half-ball lens 84 (FIG. 6A), and a BK7 glass window 86 (FIG. 6A). This lens configuration preferably forms an objective with a diffraction-limited NA of 0.71 when operating in water. The largest element, the aspheric lens 82 (FIG. 6A) has a diameter of 9.8 mm. The compact nature of these miniature lenses allows the entire objective assembly to pass through a standard PG13.5 sensor port for immersion into the bioreactor culture. The ZEMAX optical design analysis of the excitation point spread function (FIG. 6B) and the spot size across the excitation focus (FIG. 6C) are provided to demonstrate that a quasi-collimated region exists between the achromatic lens housed within the brass barrel and the aspheric lens, located within the objective. This design feature provides generous tolerance for alignment, accommodating variations in the positioning of the brass barrel and 3D-printed assembly during insertion into the stainless-steel outer tube.

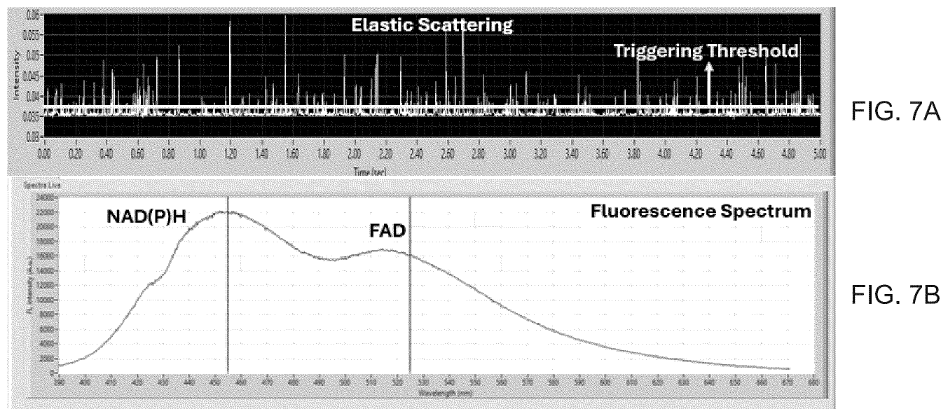

One example of the in-line spectroscopic cytometry probe measurement is the real-time monitoring of cellular redox states via NAD(P)H and FAD fluorescence. As shown in FIG. 7B, representative spectral data were collected from HEK 293 cells cultured in an agitated bioreactor. The fluorophores were excited using a 375 nm laser, though other laser wavelengths may be employed to target different intracellular fluorophores. Fluorescence signals from NAD(P)H and FAD were captured using a high-throughput spectrometer operating over a wavelength range of 380-700 nm, encompassing the emission bands of both fluorophores. Traditionally, the NAD(P)H and FAD fluorescence signals have been measured with two bandpass filters and two photodetectors. However, this approach has substantial drawbacks due to the spectral overlaps in the two fluorescence spectra. Moreover, the spectral contamination of the signal contribution from the other molecule makes the measurement inaccurate. While this impact has been dealt with stringent system calibrations, the calibrations can be compromised when other background fluorescence signals are present. In contrast, the spectroscopy approach mitigates this issue. Distinct peaks at ˜460 nm for NAD(P)H and ˜520 nm for FAD, each corresponding to individual cell emissions, clearly emerge above the background fluorescence from the culture medium. The collection of the fluorescence spectra was synchronized by the event triggers defined in the elastic scattering channel (FIG. 7A) which identifies the presence of individual cells. The event triggers were generated by applying the intensity threshold shown to the elastic scattering signal peaks.

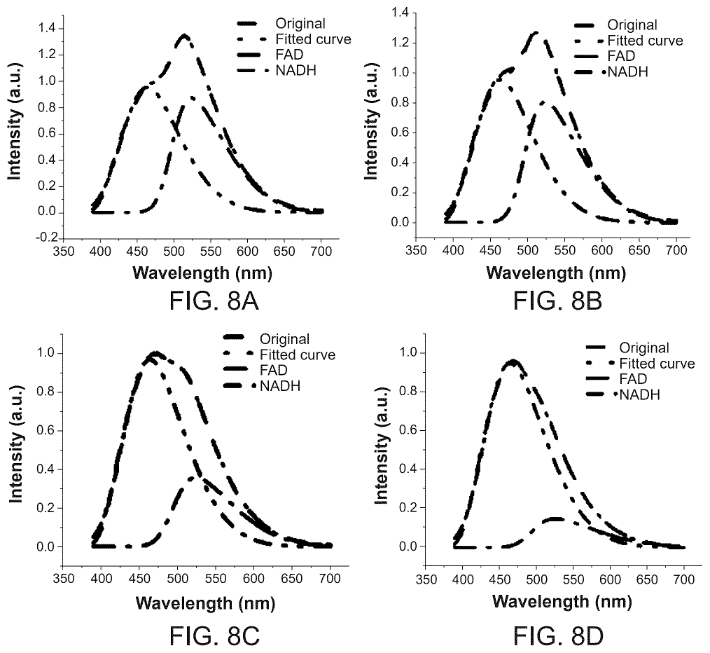

To differentiate NAD(P)H and FAD signals shown in FIG. 7B, a spectroscopic data processing algorithm based on skewed Gaussian fitting (SGF) may be employed. In this method, the measured fluorescence spectra are decomposed using two skewed Gaussian functions, each representing one of the fluorophores. As a validation example, the SGF method was applied to mixed solutions of NAD(P)H and FAD at varying concentrations. The NADH concentration was held constant at 1000 μmol, while the FAD concentration was varied at 50 (FIG. 8A), 25 (FIG. 8B), 12.6 (FIG. 8C), and 6.3 (FIG. 8D) μmol to evaluate the algorithm. The resulting processed data, shown in FIGS. 8A-8D. This data demonstrates the ability of the algorithm to efficiently separate NAD(P)H and FAD spectral components across a range of concentrations. This SGF-based spectral analysis approach significantly enhances the accuracy and efficiency of signal separation compared to conventional methods, such as the bandpass filter technique.

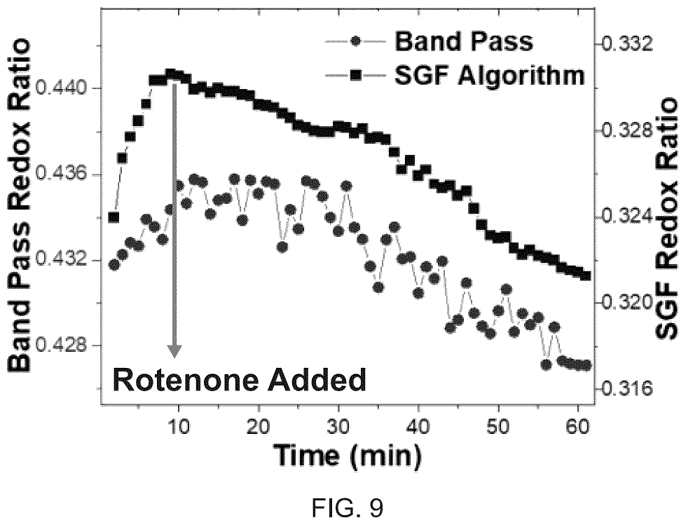

The in-line spectroscopic cytometry data offers rich information regarding the cell physiology status and culture conditions. In particular, the cellular-level redox metabolism can be calculated based on the NAD(P)H and FAD fluorescence. FIG. 9 shows the cellular redox changes of HEK 293 cell culture before and after the addition of rotenone into the cell culture at approximately 10 minutes as indicated by the arrow. Rotenone inhibits the mitochondrial electron transport chain by inhibiting the oxidation of NAD(P)H in cells leading to an increase in NAD(P)H concentration. NAD(P)H accumulation leads to a decrease in the redox ratio. The two curves in FIG. 9 illustrate the results using the two different data analysis methods. The band pass approach applied two bandpass filters to the spectra to emulate the measurement methodologies using two spectral filters which provides much noisier results. The SGF Algorithm curves indicate the results using the spectral fitting methods based on skewed Gaussian models, which showed much higher SNR, demonstrating the advantages of the event-triggered spectroscopy approach.

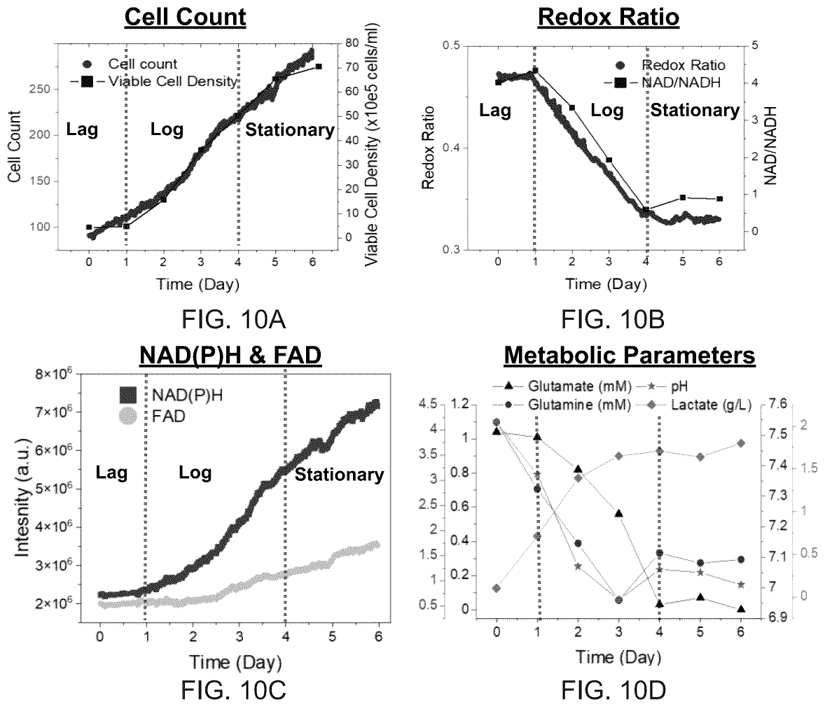

FIGS. 10A-10D present an example of continuous redox metabolism monitoring during a growth run of HEK 293 cell culture in a bioreactor performed without viral transfection and using the in-line spectroscopic cytometry probe system. The figures demonstrate key parameters measured by the spectroscopic cytometry sensor including cell count (FIG. 10A), NAD(P)H and FAD fluorescence levels (FIG. 10C), and the derived redox ratio (FIG. 10B). The probe sensor-derived redox ratio and cell count data were validated by comparison with standard offline reference measurements. The bioreactor-cultured HEK cells exhibited the expected three-phase growth profile: lag, exponential (log), and stationary phase. These phases were clearly reflected in the measured parameters, including cell count, NAD(P)H and FAD levels, and redox ratio trends. The probe-sensor measured a consistent increase in cell count over the operation period, aligning well with daily viable cell density (VCD) data acquired from extracted samples, though the offline method offered lower time resolution. Although cell viability was not measured directly, resulting in a slight deviation between cell count and VCD, the trends remained consistent, supporting the cell counting capability of the sensor, FIG. 10A.

FIG. 10B includes a comparative analysis between the in-line redox ratio and data obtained from offline NAD/NADH quantification assays. To validate the phase variations, offline NAD/NADH assays were performed, with NAD⁺ used as a surrogate marker for FAD. The trends from these offline assays were consistent with the in-line measurements confirming the effectiveness of the in-line cytometry probe in monitoring redox dynamics during cell culture.

Throughout the process, NAD(P)H levels increased at a faster rate than FAD, suggesting elevated energy demands during cell expansion and metabolic activation. The redox ratio, derived from the relative fluorescence intensities of NAD(P)H and FAD, clearly delineated the different growth phases. It remained relatively constant during the lag phase, decreased during the exponential phase, indicating heightened metabolic activity, and then stabilized during the stationary phase, reflecting a metabolic shift toward a more reductive state, FIG. 10C.

In addition to fluorescence-based redox measurements, cellular metabolites such as lactate, glutamine, glutamate, and pH were monitored during the growth runs. These metabolite trends were consistent with redox dynamics observed by the cytometry sensor, FIG. 10D.

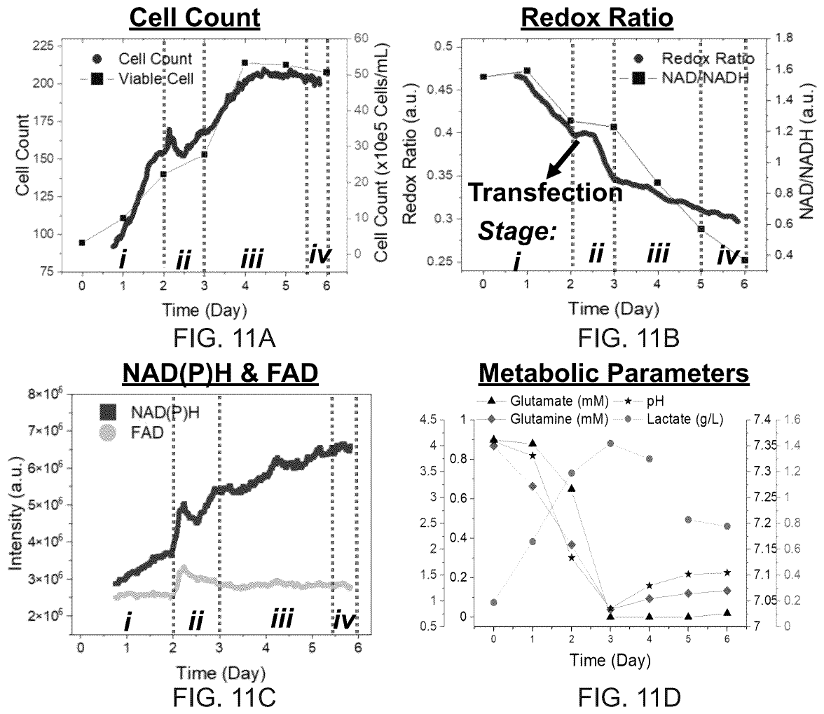

FIGS. 11A-11D present an example of another continuous redox metabolism monitoring with viral transfection during a growth run of HEK 293 cell culture in a bioreactor. The transfection process utilized the AAV2 serotype and was initiated in a bioreactor. As shown in FIG. 11A, cell counts increased steadily prior to transfection (stage i), fluctuated during the transfection phase (stage ii), resumed a gradual rise post-transfection (stage iii), and eventually stabilized (stage iv). These trends were in agreement with both NAD(P)H and FAD fluorescence levels (FIG. 11C) and offline viable cell density (VCD) measurements. Owing to its high temporal resolution, the in-line probe sensor captured significant short-term fluctuations during the transfection phase which are likely driven by metabolic stress, the cellular response to foreign DNA introduction, and increased protein production demands.

These redox ratio changes were consistent with data obtained from offline NAD/NADH assays (square data points connected by lines in FIG. 11B), which support the redox measurement capability of continuous in-line cytometry to resolve dynamic changes observed in real time. The continuous monitoring capability enabled detection of these metabolic transitions, offering valuable insight into cellular adaptation during transfection, FIG. 11B. Complementary offline analyses of culture metabolites, glutamine, glutamate, lactate, and pH, were also performed during the transfection run (FIG. 11D).

Although specific features of the invention are shown in some drawings and not in others, this is for convenience only as each feature may be combined with any or all of the other features in accordance with the invention. The words “including”, “comprising”, “having”, and “with” as used herein are to be interpreted broadly and comprehensively and are not limited to any physical interconnection. Moreover, any embodiments disclosed in the subject application are not to be taken as the only possible embodiments. Other embodiments will occur to those skilled in the art and are within the following claims.

In addition, any amendment presented during the prosecution of the patent application for this patent is not a disclaimer of any claim element presented in the application as filed: those skilled in the art cannot reasonably be expected to draft a claim that would literally encompass all possible equivalents, many equivalents will be unforeseeable at the time of the amendment and are beyond a fair interpretation of what is to be surrendered (if anything), the rationale underlying the amendment may bear no more than a tangential relation to many equivalents, and/or there are many other reasons the applicant cannot be expected to describe certain insubstantial substitutes for any claim element amended.

Other embodiments will occur to those skilled in the art and are within the following claims.

What is claimed is:

1. An in-situ cytometry method of monitoring cell status and reducing background fluorescence and/or background scattering signals from non-cellular material, the method comprising:
directing electromagnetic energy into a cytometry volume, located within an agitated bioreactor or fermenter, said cytometry volume being subcellular in size, to stimulate elastic scattering photons and fluorescence and/or Raman photons from a cell present in the cytometry volume;
directing elastic scattering photons from the agitated bioreactor or fermenter to a detector which outputs a trigger signal;
directing fluorescence and/or Raman photons from the agitated bioreactor or fermenter to a spectroanalysis subsystem; and
selectively spectroanalyzing the fluorescence and/or Raman photons in response to the trigger signal output by the detector to determine a biochemical profile of the cell and to reduce background noise.

2. The method of claim 1 in which selectively spectroanalyzing the fluorescence and/or Raman photons includes not allowing the fluorescence and/or Raman photons to reach the spectroanalysis subsystem until the trigger signal is output by the detector.

3. The method of claim 1 in which the spectroanalysis subsystem includes a spectrometer and a processing subsystem responsive to an output of the spectrometer, the processing subsystem triggered by the trigger signal to spectroanalyze the fluorescence and/or Raman photons.

4. The method of claim 1 in which the fluorescence photons are from NAD(P)H and FAD intracellular metabolites.

5. The method of claim 4 further including differentiating between NAD(P)H and FAD intracellular metabolites.

6. The method of claim 5 further including calculating a fluorescence ratio FAD/(FAD+NAD(P)H) to quantify a fluorescence redox ratio.

7. The method of claim 6 further including quantifying the redox ratio using a spectral band pass algorithm and/or a skewed Gaussian fitting algorithm.

8. An in-situ cytometry system for monitoring cell status and reducing background fluorescence and/or background scattering signals from non-cellular material, the system comprising:
a probe insertable into an agitated bioreactor or fermenter, the probe defining a subcellular cytometry volume in the bioreactor or fermenter;
a source outputting electromagnetic energy to the probe to stimulate elastic scattering photons and fluorescence and/or Raman photons of a cell present in the cytometry volume;
a first detection channel directing elastic scattering photons from the probe to a detector which outputs a trigger signal;
a second detection channel directing fluorescence and/or Raman photons from the probe to a spectrometer; and
means for selectively spectroanalyzing the fluorescence and/or Raman photons in response to the trigger signal from the detector to determine a biochemical profile of the cell and to reduce background noise.

9. The system of claim 8 in which the means for selectively spectroanalyzing includes a shutter configured to allow the fluorescence and/or Raman photons to reach a spectroanalysis subsystem in response to the trigger signal.

10. The system of claim 9 in which the shutter includes a DMD.

11. The system of claim 8 in which the means for selectively spectroanalyzing includes a processing subsystem responsive to an output of a spectrometer, the processing subsystem triggered by the trigger signal to determine a biochemical profile of the cell.

12. The system of claim 8 further including a processing subsystem configured to determine a fluorescence redox ratio of the cell from the fluorescence photons.

13. The system of claim 12 in which the fluorescence scattering photons are from NAD(P)H and FAD intracellular metabolites.

14. The system of claim 13 in which the processing subsystem is configured to differentiate between NAD(P)H and FAD intracellular metabolites.

15. The system of claim 14 in which the processing subsystem is configured to calculate a fluorescence ratio FAD/(FAD+NAD(P)H) to quantify a fluorescence redox ratio.

16. The system of claim 15 in which the processing subsystem is configured to quantify the redox ratio using a spectral band pass algorithm and/or a skewed Gaussian fitting algorithm.
